(19) United States
(12) Patent Application Publication
(10) Pub. No.: US 2016/0000379 A1
(43) Pub. Date: Jan. 7, 2016

(54) METHOD AND APPARATUS FOR DYNAMIC ASSESSMENT AND PROGNOSIS OF THE RISKS OF DEVELOPING PATHOLOGICAL STATES

(71) Applicants: Vadim Ivanovich Pougatchev, Poulsbo, WA (US); Roman Markovich Baevskiy, Moscow (RU); Anna Grigorievna Chernikova, Moscow (RU); Oleg Igorevich Orlov, Moscow (RU); Alexei Romanovich Baevski, Mississauga (CA); Olga Nikolaevna Isaeva, Vladimir (RU)

(72) Inventors: Vadim Ivanovich Pougatchev, Poulsbo, WA (US); Roman Markovich Baevskiy, Moscow (RU); Anna Grigorievna Chernikova, Moscow (RU); Oleg Igorevich Orlov, Moscow (RU); Alexei Romanovich Baevski, Mississauga (CA); Olga Nikolaevna Isaeva, Vladimir (RU)

(21) Appl. No.: 14/320,751

(22) Filed: Jul. 1, 2014

(51) Int. Cl. A61B 5/00; A61B 5/024; A61B 5/0456; A61B 5/0245

(52) U.S. Cl. CPC A61B 5/7275; A61B 5/0245; A61B 5/02416; A61B 5/02405; A61B 5/0456

(57) ABSTRACT

A method and apparatus for health risk assessment by means of measuring and evaluating the functional state of the mechanisms of physiological adaptation of human organism comprises recording analog signals of heartbeat intervals, filtering the analog signals of heartbeat intervals, converting the analog signals to digital signals of heartbeat intervals, transmitting the digital signals of heartbeat intervals to a central processing unit, detecting R-peaks from the digital signals of heartbeat intervals, measuring time intervals between two consecutive said R-peaks, calculating heartbeat variability parameters from said heartbeat intervals, calculating degree of tension (DT) of regulatory mechanisms and their functional reserve (FR) based on said heartbeat variability parameters, calculating plurality of functional states based on the DT and FR, and calculating posteriori probabilities based on plurality of functional states, wherein probabilities of health risk is determined based on values of DT, FR, at least one functional state and the posteriori probabilities.

### FIELD OF THE INVENTION

The present invention generally relates to health risk assessment. More particularly, the present invention relates to a method and apparatus for dynamic assessment and prognosis of risks of developing pathological states.

### BACKGROUND OF THE INVENTION

The concept of home health is one of the serious trends in healthcare and technology nowadays. It is aimed to provide ordinary people with simple means to assess, monitor and maintain various aspects of their personal health as well as prevent health problems. Till recent time thermometers, cuff blood pressure monitors and glucose meters were the most common home health devices. Later various kinds of simple wearable heart rate monitors appear mainly used by young athletes in their workout programs. Among them there was the Heart Wizard developed by Biocom Technologies (USA) taking a special niche. It provides not only ability to monitor heart rate but number of special health tests based on heart rate variability (HRV) analysis known as a strong means to access autonomic regulation of cardiovascular system.

When Russian special space program called “Mars 500” was developed to model a long-term flight to Mars, the Heart Wizard was chosen to monitor health trends in volunteers during 18-month study. This tool was specially adapted for the study by Canadian and Russian scientists and extended with new experimental software components and then successfully used in the study on US and Canadian volunteers in 2009-2012. In addition to the standard pulse wave sensor used for conducting standard HRV tests, special health questionnaire was added to collect biometrics and important health information such as blood pressure. Also new unique algorithms of analysis of health information developed for space medicine using pre-nosological health evaluation model were also added.

Use of the Heart Wizard in Mars 500 project for long-term tracking the health parameters of healthy volunteers revealed increased risks of disease development caused by lifestyle, seasonal weather changes and growing stressors. These findings lead to developing a new instrument called “Delta” for personal pre-nosological health assessment based on dynamic evaluation of health risks caused by possible changes in mechanisms of physiological adaptation of the body (adaptation risk). Adaptation risk is a new term in physiology and medicine introduced in space medicine for quantitative assessment of the risks of developing new health disorders in healthy individuals. There were 10 risk categories established with gradually increasing risks of developing pathologies based on current functional state of the organism.

Further analysis of technologies and instruments for individual home health available in the market is outlined below. Beginning 2008 a new diagnostic instrument “Angioscan-01” was approved for medical market in Russia. This device records pulse wave with photoplethysmograph (PPG) sensors placed on index fingers of both hands for further evaluation of endothelial function of peripheral blood vessels. The sensors continuously record pulse wave. Using an arm cuff the device creates occlusion of the right brachial artery by generating pressure level 50 mmHg over systolic level. Then endothelial function is accessed using an original algorithm.

Another useful home health device is Pulsewave™ MAX from Biosign Series. It includes four components: blood pressure cuff, blood oxygen sensor, high fidelity thermometer and ECG. The device measures four main physiological signals (sphygmograph, blood oxygen saturation, body temperature and ECG) and six additional parameters using their proprietary Cloud Diagnostics™ algorithm. The device is battery powered providing wireless link with a PC, tablet or smartphone to feed live data or log data in its flash memory. The device is suitable for express diagnostics or live monitoring in critical care. Biosign product line offers a special smartphone application Pulsewave™ 3.0 to collect and analyze pulse wave data from various fitness and medical devices.

Another interesting instrument is emWave® Health Professional. It provides patients with home tools to recover after illness without frequent visits to doctors and track their health changes individually at home. It includes the software, ear-clip USB sensor and standalone biofeedback device. It also provides access to health data for professionals for review and consultations over the Internet.

MaxPulse is another home health device from The Wellness and Aesthetics (USA). This device is interfaced with Windows PC and measures pulse wave signal using a PPG technology. It analyzes pulse waveform and heart rate variability. The sensor has an ear-clip form factor. The device measures general elasticity of large and small peripheral arteries, functional vascular age, hydration level, heart rate and left ventricle ejection fraction. This device may be utilized by health professionals for early detection of cardiovascular disorders, measuring the effects of drugs and optimization of their administration.

The majority of the wearable home health devices emerging for the past 5-6 years are focused on comfortable and ergonomic ways of gathering health information, which is very important aspect of home health tools. The most popular are different kinds of bracelets with embedded PPG sensors or ECG electrodes. There are new kinds of devices for long-term recording of ECG signal using the electrodes embedded in clothes called “smart clothes”. They usually utilize “dry” electrodes made off conductive textile fibers weaved in the fabrics. These kinds of electrodes however produce higher levels of signal disturbances caused by movement or even breathing. One of such textile ECG electrodes is offered by “Equivital” company from the UK. Their electrodes are embedded in a textile chest strap with one brace over left shoulder. There was a special study conducted in Korea to find the most optimal position for such textile electrodes. The study showed that the most appropriate placement method is to position textile electrodes crosswise over the chest.

An Italian company Smartex produces t-shirts with embedded textile ECG electrodes for single-channel ECG recording. Conductive fibers are weaved in the fabric at specific locations. They consist of thin metal wires twisted with cotton and synthetic threads. An ECG recorder is placed in a special pocket located on the chest. Similar products have additional electrodes embedded in the fabric for recording of breathing activity.

Smart Life, a British company has announced their smart clothes with embedded dry sensors capable of recording ECG, heart rate, EMG, respiration and body temperature. These clothes are made of double-layer threads with weaved in synthetic fibers covered with silver. The company claims the capability to record ad transmit bio-signals to a remote PC or smartphone via Bluetooth link. The data can be logged into flash memory embedded in the clothes.

For the past few years non-contact electrodes were developed as an alternative to textile electrodes. They measure changes in body surface capacitance instead of measuring changes in voltage. Their main advantage is the ability to measure bio-signals through the layers of clothes. This makes possible to embed electrodes in chairs.

Non-contact electrodes could be used in home health tools for sleep monitoring. One of the most effective devices using these principles is “Sonocard” developed in Russia and utilized on board of the International Space Station (ISS). This device has size of a pack of cigarettes wearable in a chest pocket. It includes 3-D accelerometer, amplifier, flash memory and battery. During the night it records chest oscillations caused by heartbeats (seismocardiogram). After waking up it downloads the recorded data to the on-board computer which transmits the data to the control center for further analysis. The device was used on ISS for 5 years and provided enormous amount of data about crew members' sleep. Earlier the crew members of the orbital station “Mir” were using special sleeping bags with embedded seismocardiograph sensors.

Based on the results of physiological studies conducted on space stations a new medical device “CardioSleep 3” was developed in Russia and was used in research and clinical studies. This device is placed under sleeping mattress or pillow in patient bed. It consists of two light metal plates with a pressure sensor between them detecting heart pulsations. Measured pressure signal is set to a remote PC or stored in flash memory.

Recently Carré Technologies, a Canadian company introduced a “smart shirt” for physiological recording in space. It also has embedded dry electrodes. Currently this device is being used in one of the Antarctic expeditions. This company also designed a commercial product Hexoskin for sports and sleep studies applications.

For the past few years there is a strong trend in showing up various kinds of individual home health applications for mobile devices utilizing new technologies similar to those developed for space medicine. Finally it is worth to mention that booming industries of home health, sports and fitness are mainly based on traditional methods and technologies used in mainstream medical practice despite of new emerging technologies. We believe that the next breakthrough in home health technologies will be coming from the new concept of pre-nosological health assessment originally developed for experimental space medicine.

At this time there are no reliable means in consumers' possession capable to effectively monitor and preserve their health condition. Typically people seek medical help when symptoms become apparent and disease is progressing. It is still fairly uncommon when people visit doctors for regular health checkup to prevent possible disease or at least detect health problems at early stage. Even in such cases doctors often check for presence of any physical, biochemical or physiological signs of disease. Although it is a known fact that most of symptoms become apparent not at early stage of the disease due to extremely high ability of the human organism to adapt and compensate any moderate dysfunction. In fact, a lot of changes occur in the mechanisms of body adaptation at earlier stages of the disease before specific symptoms appear. Learning about negative changes in adaptive capabilities of an organism caused by pathogens helps to determine whether the disease process is underway before the first known symptoms become evident. Such changes may be identified as borderline or preclinical (also known as premorbid) states as precursors of the developing pathologies.

Consequently, there exists in the art a long-felt need for a method and apparatus for assessment of adaptation capabilities of a human organism and risks of disease development. There also exists in the art a long-felt need for a method and apparatus which is both for professional and personal use. Finally, there is a long felt need in the art for a method and apparatus that accomplishes all of the forgoing objectives and that is relatively inexpensive to manufacture and install, safe, easy to use and implement, and is extremely time saving.

### BRIEF SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Accordingly, the present invention provides a method of quantitative assessment of risks of developing borderline, premorbid and pathological states by means of measuring and evaluating the functional state of the mechanisms of physiological adaptation of the human organism.

According to one embodiment of the present invention, the method may utilize recording of electrocardiograph (ECG) or photoplethysmograph (PPG) signal at rest for a specific period of time, such as for 5 minutes, for less than 5 minutes or for more than 5 minutes using respective recording devices interfaced with a personal computer.

In another embodiment of the present invention, recorded ECG or PPG signals are analyzed using specific known algorithms to measure beat-by-beat heartbeat intervals.

In yet another embodiment of the present invention, the obtained series of heartbeat intervals representing 5-minute, less than 5 minute or more than 5 minute signal recording are processed with known algorithms of heart rate variability (HRV) analysis to calculate specific parameters: heart rate (HR), stress index (SI), pNN50 and high frequency percentage ratio (HF %). These parameters are used to calculate the degree of tension of regulatory mechanisms (DT) and their functional reserve (FR) using canonical discriminant function equations.

The obtained values of DT and FR are used in the original manner to calculate the values of respective discriminant functions and identify four types of states: normal, borderline, premorbid and pathological.

In still another embodiment of the present invention, the obtained values of the discriminant functions are submitted to the equations for calculation of posterior probabilities of risks of developing respective states: normal, borderline, premorbid and pathological.

The results of this assessment may be visually presented in the form of a special diagram with orthogonal coordinates of DT versus FR. In one preferred embodiment of the present invention, the plot area of this diagram is divided by two orthogonal axes into four quadrants defining those four states: normal, borderline, premorbid and pathological state. In an exemplary embodiment of the present invention, a resulting dot is placed on the diagram in accordance with the respective values of DT and FR obtained from the measured data indicating the current condition of the mechanisms of physiological adaptation.

In another preferred embodiment of the present invention, the obtained values of probabilities of risk of developing four respective states may be presented in the form of proportional bar diagram. Each probability of respective state may be presented in a specific color intuitively associated with the respective state.

When multiple testing based on this method is performed over time, the evaluation of the dynamics of changes in condition of the adaptation mechanisms and risks of developing respective states may be done. Such dynamics is evaluated using standard methods of regression analysis. The obtained results of this analysis may allow for making conclusions about the dynamics of changes taking place—their degree and direction. Depending on the number of assessments performed over time the level of statistical significance of such analysis can be estimated.

Based on the results of such regression analysis, mathematical time forecast of the estimated variables may be obtained using the methods of mathematical extrapolation of an appropriate order. Also confidence values may be obtained for such forecast.

To the accomplishment of the foregoing and related ends, certain illustrative aspects of the disclosed invention are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a normal ECG signal and a way to process it to obtain heartbeat intervals;

FIG. 2 illustrates methods of ECG electrode placement and principles of technical implementation of ECG measurement and data transfer to an external computing device;

FIG. 3 illustrates a method of ECG measurement and calculation of heartbeat intervals by means of a standalone wearable device and its data transmission to an external computing device via a wireless communication channel;

FIG. 4 illustrates a normal PPG signal and a way to process it to obtain heartbeat intervals;

FIG. 5A illustrates how a PPG sensor works using penetration of perfused tissues with infrared light;

FIG. 5B illustrates how a PPG sensor works using penetration of perfused tissues with reflection of infrared light;

FIG. 6 illustrates methods of PPG sensor placement and principles of technical implementation of PPG measurement and data transfer to an external computing device;

FIG. 7A illustrates the suggested method of presentation in orthogonal quadrants the results of assessment of the condition of human adaptation mechanisms and risks of developing of pathological states being implemented in ordinary software products;

FIG. 7B illustrates the suggested method of presentation of the results of assessment of the condition of human adaptation mechanisms and risks of developing of pathological states being implemented in ordinary software products in form of bar diagram;

FIG. 8 illustrates a generic structure of the computer-based system performing this method of assessment of adaptation capabilities of human organism and risks of disease development;

FIG. 9A illustrates a wristwatch form for implementation of a computer-based system performing this method of assessment of adaptation capabilities of human organism and risks of disease development;

FIG. 9B illustrates a bracelet, a chest strap, an ear clip forms for implementation of a computer-based system performing this method of assessment of adaptation capabilities of human organism and risks of disease development;

FIG. 9C illustrates a device physically connected to a smart phone for implementation of a computer-based system performing this method of assessment of adaptation capabilities of human organism and risks of disease development;

FIG. 9D illustrates a device with an enclosure for implementation of a computer-based system performing this method of assessment of adaptation capabilities of human organism and risks of disease development;

FIG. 9E illustrates a kiosk form for implementation of a computer-based system performing this method of assessment of adaptation capabilities of human organism and risks of disease development;

FIG. 10 and FIG. 11 illustrate an exemplary flow chart for implementing the method of assessment of adaptation capabilities of human organism and risks of disease development;

### DETAILED DESCRIPTION OF THE INVENTION

The following description is presented to enable any person skilled in the art to make and use the invention, and is provided in the context of particular applications of the invention and their requirements. Various modifications to the disclosed embodiments will be readily apparent to those skilled in the art and the general principles defined herein may be applied to other embodiments and applications without departing from the scope of the present invention. Thus, the present invention is not intended to be limited to the embodiments shown, but is to be accorded the widest scope consistent with the principles and features disclosed herein.

In the following detailed description, numerous specific details are set forth in order to provide a thorough understanding of the invention. However, it will be understood by those skilled in the art that the present invention may be practiced without these specific details. In other instances, well-known methods, procedures and components have not been described in detail so as not to obscure the present invention.

Unless specifically stated otherwise, as apparent from the following discussions, it is appreciated that throughout the specification discussions utilizing terms such as “processing”, “computing”, “determining”, “calculating” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

Embodiments of the present invention are described herein in the context of a method and apparatus for dynamic assessment and prognosis of the risks of developing pathological states. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

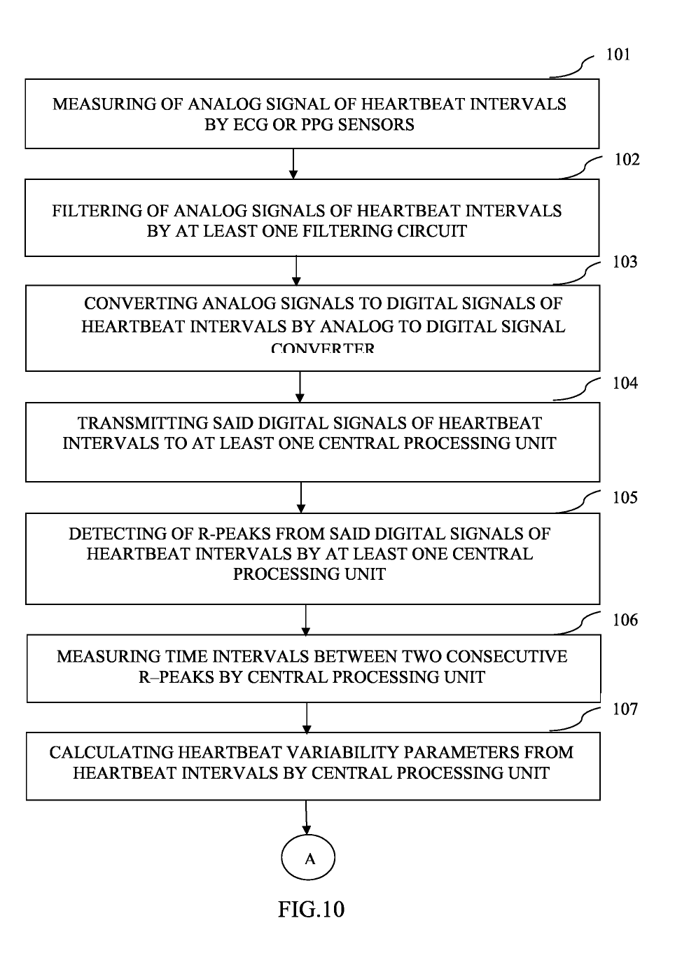

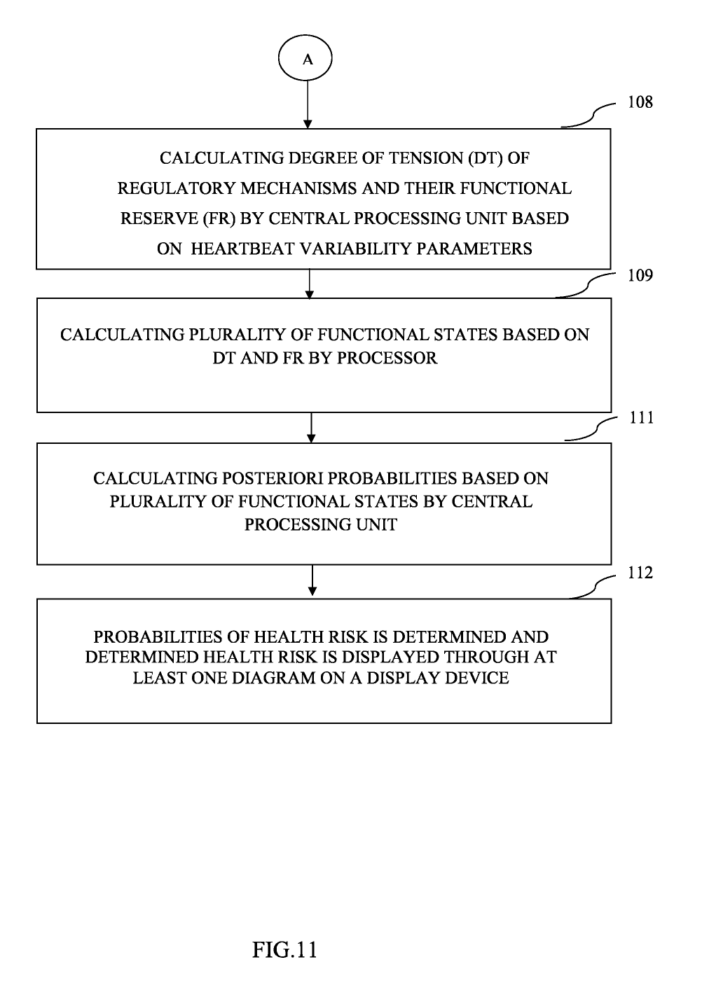

Flowcharts are used to describe the steps of the present invention. While the various steps in these flowcharts are presented and described sequentially, some or all of the steps may be executed in different orders, may be combined or omitted, and some or all of the steps may be executed in parallel. Further, in one or more of the embodiments of the invention, one or more of the steps described below may be omitted, repeated, and/or performed in a different order. In addition, additional steps, omitted in the flowcharts may be included in performing this method. Accordingly, the specific arrangement of steps shown in FIGS. 10 and 11 should not be construed as limiting the scope of the invention.

The present invention provides a method of quantitative assessment of risks of developing borderline, preclinical (premorbid) and pathologicalstates by means of measuring and evaluating functional state of the mechanisms of physiological adaptation of human organism and generating respective conclusion based on the results of such assessment.

In one embodiment of the present invention, the method includes and is based upon a procedure of recording of a series of heartbeat intervals at rest for a specific period of time, such as for 5 minutes, for less than 5 minutes and for more than 5 minutes.

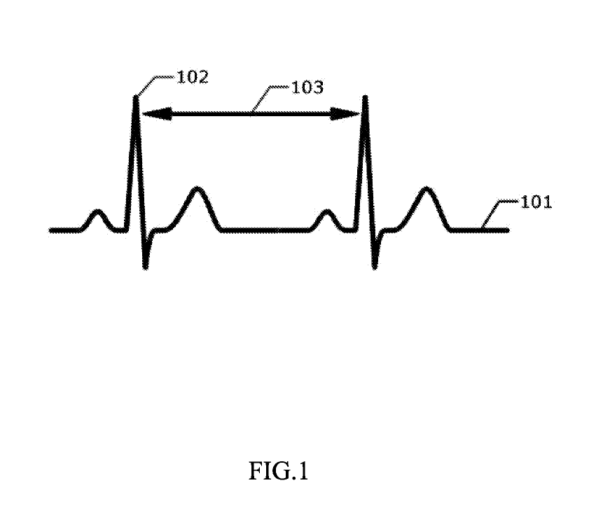

As best illustrated in FIG. 1, heartbeat interval recording may be carried out by means of measuring electrocardiograph (ECG) signal [101] with respective ECG signal processing using a special R-peak [102] detection algorithm and calculation of time intervals [103] between consecutive R-peaks in accordance with one embodiment of the present invention.

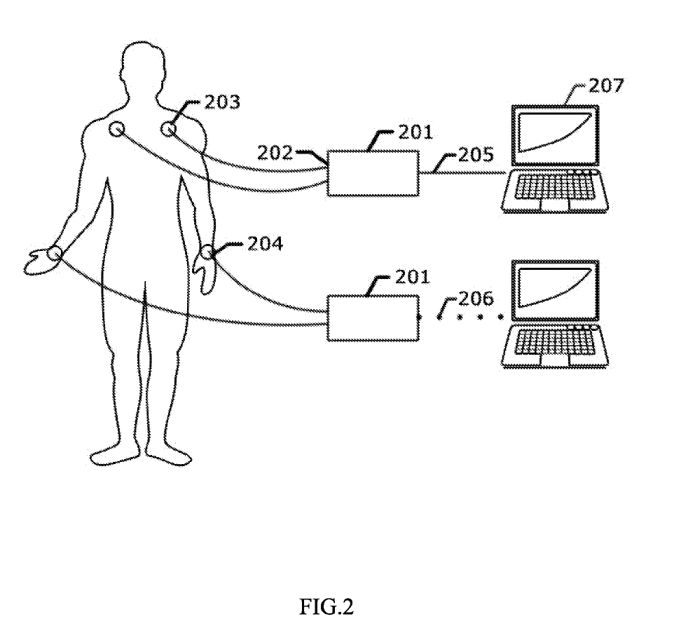

As best illustrated in FIG. 2, ECG recording may be carried out by means of a special device—ECG recorder [201] with at least one input channel [202] connected to at least two ECG sensors or electrodes placed on the subject's skin in accordance with standard schemes of ECG electrode placement and ECG signal recording. Two ECG electrodes may be placed on wrists or forearms of both arms [204] or on chest to the left and right of the heart area [203] in accordance with one embodiment of the present invention. Other standard ways of ECG electrode placements are possible too. In another embodiment of the present invention, measured ECG signal is converted in digital format by means of respective electronic circuit and further transmitted to a computer [207] via a wired (e.g. USB) [205] or wireless (e.g. Bluetooth, WiFi) [206] channel.

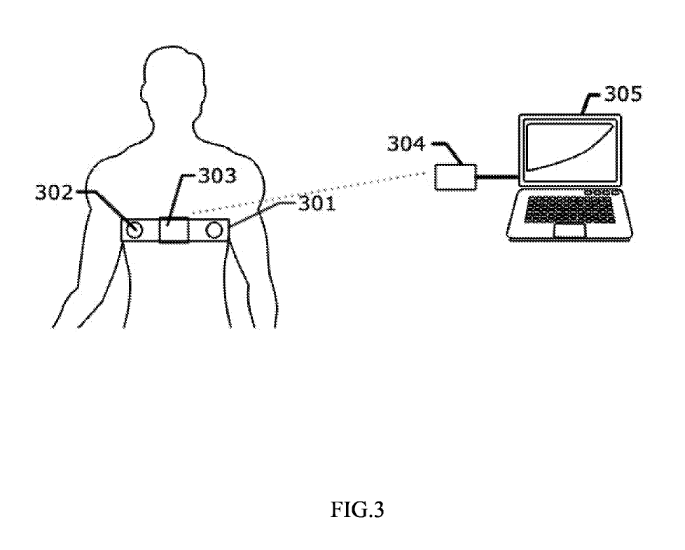

As best illustrated in FIG. 3, ECG recording may also be carried out by means of a standard chest strap sensor [301] with two built-in electrodes [302] made of conductive rubber, stainless steel or other appropriate conductive material in accordance with one embodiment of the present invention. Such chest strap [301] may include an electronic circuitry [303] carrying out ECG signal measurement, R-peak detection, generation of square waves representing detected R-peaks and wireless transmission of those waves modulated in an appropriate radio signal. A respective radio receiver [304] reads this signal, converts it into digital format and sends to a computer [305].

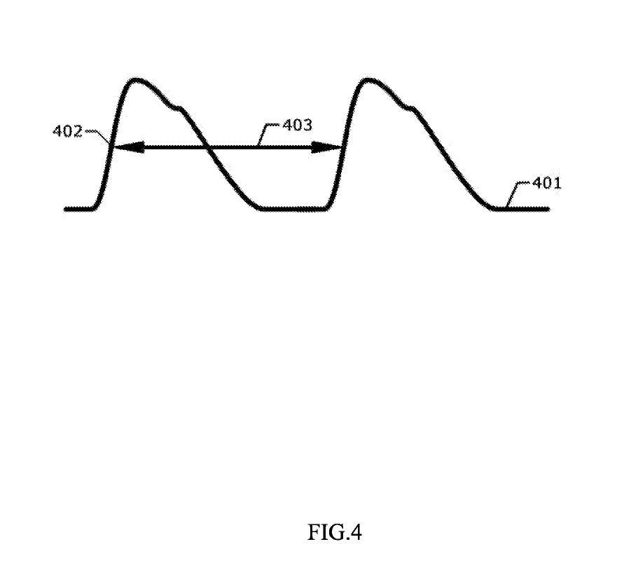

As best illustrated in FIG. 4, heartbeat interval recording may be carried out by means of measuring a photoplethysmograph (PPG) signal [401], its processing with an appropriate algorithm of pulse wave [402] detection and calculation of time intervals between appropriate fiduciary points on consecutive pulse waves [403]. Such fiduciary points could be the points of maximum ascending slope of the pulse wave [402].

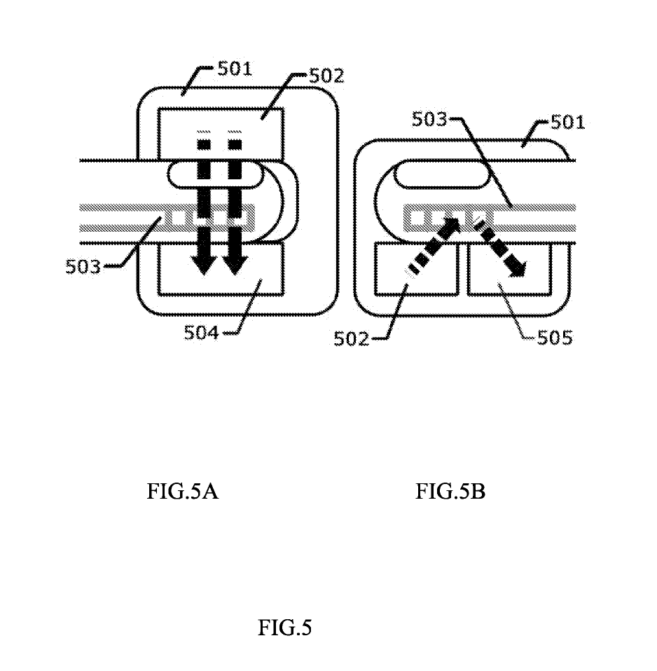

As best illustrated in FIG. 5A and FIG. 5B, in accordance with one embodiment of the present invention, PPG recording may be carried out by means of a special device—PPG sensor [501]. PPG sensors utilize a principle of absorption of infrared light by hemoglobin molecules. One part of the sensor includes the source of infrared light [502] emitting the IR light toward the tissues penetrated with blood capillaries [503]. The other part of the sensor includes an IR light receiver [504, 505] placed in such a way to catch IR light either passing through the tissue [504] or reflected from that tissue not absorbed by hemoglobin of the blood. Since the amount of blood passing through the capillaries lighted up with IR light emitted from the IR light source continuously changes over time due to pulsatile nature of the blood flow, the amount of IR light passed through or reflected from the tissues also changes proportionally to the amount of blood passing through the tissues. As a result of this the IR receiver [504 or 505] generates an electrical signal having a specific pulse waveform.

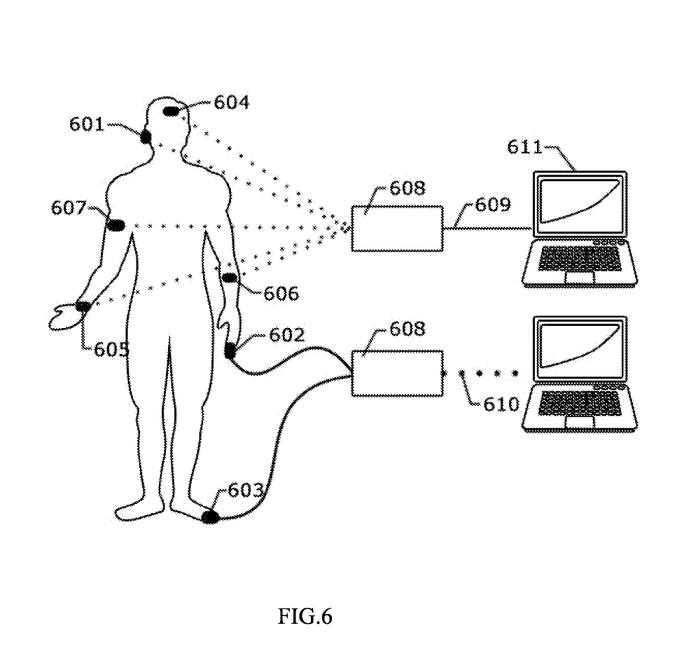

As best illustrated in FIG. 6, a PPG sensor of respective type may be placed in various areas of the human body. In an exemplary embodiment of the present invention, pass-through type sensors may be placed on earlobes [601], fingers [602] or toes [603]. Reflection type sensors may also be placed on earlobes, fingers or toes as well as on forehead [604], wrists [605], forearms [606], arms [607] and other locations where the sensor can be securely placed to avoid its displacement during signal recording. In another exemplary embodiment of the present invention, measured PPG signal may be converted into digital format by means of a respective electronic circuit [608] and then transmitted to a computer [611] via a wired (e.g. USB) [609] or wireless (e.g. Bluetooth, WiFi) [610] channel.

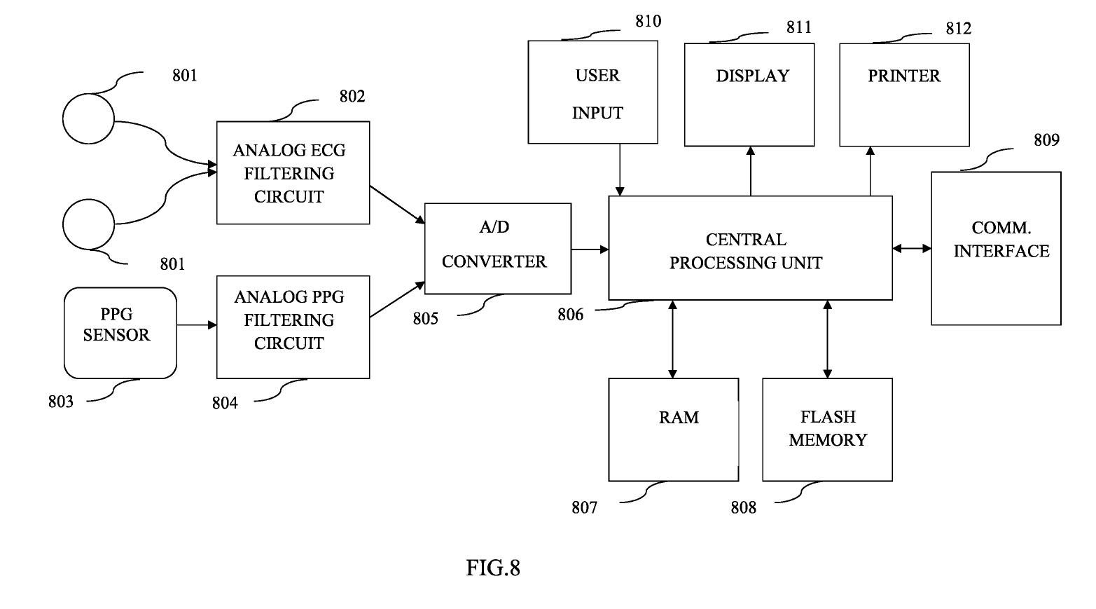

In a preferred embodiment of the present invention, reference to FIG. 8, upon completion of measuring or recording of ECG or PPG signal for a specific period of time and their respective processing with regard to measuring heartbeat intervals by means of respective algorithms of R-peak or pulse wave detection, a set of standard known heart rate variability (HRV) parameters is calculated by the central processing unit [806].

The following HRV parameters are mandatory to calculate: (a) Heart Rate (HR), (b) Stress Index (SI), (c) pNN50, (d) HF %.

HR is a mean value of all HR values calculated off normal heartbeat intervals detected preferably within 5-min recording.

SI is an index of stress level also known as stress index of regulatory systems calculated using the following formula:

$$SI = \frac{AMo}{2 Mo Δ X}$$

where,
AMo—is an amplitude of mode, which is the total number of heartbeat intervals corresponding to the mode value calculated as percentage of the total number of heartbeat intervals.
Mo—mode, the most frequently observed heartbeat interval value in a series of recorded heartbeat intervals measured in seconds.
ΔX—range (the difference between maximal and minimal values) of a series of recorded heartbeat intervals measured in seconds.
pNN50—percentage of the number of consecutive pairs of normal heartbeat intervals having difference of 50 or more milliseconds to the total number of consecutive pairs of normal heartbeat intervals of the recorded series.
HF % —a percentage ratio of the spectral power of normal heartbeat intervals in the high frequency (HF) range to the total spectral power of normal heartbeat intervals (TP) calculated using the following formula:

$$HF \% = 100 \times \frac{HF}{TP}$$

where,
TP—the sum of spectral powers of normal heartbeat intervals calculated in all frequency ranges using the following formula:

TP=VLF+LF+HF

where,
VLF—spectral power of normal heartbeat intervals calculated in very low frequency range Δf_{VLF} (0.0033 Hz-0.04 Hz) using the following formula:

$$VLF = {\sum }_{j = {L}_{VLFl}}^{{L}_{VLFr}} {P}_{j} , ( {ms}^{2} )$$

where L_{VLFl} and L_{VLFr}—the frequency limits of the very low frequency range Δf_{VLF} and Pⱼ—spectral values within this frequency range;
LF—spectral power of normal heartbeat intervals calculated in low frequency range Δf_{LF} (0.04 Hz-0.15 Hz) using the following formula:

$$LF = {\sum }_{j = {L}_{LFl}}^{{L}_{LFr}} {P}_{j} , ( {ms}^{2} )$$

where L_{LFl} and L_{LFr}—the frequency limits of the low frequency range Δf_{LF} and Pⱼ—spectral values within this frequency range;
HF—spectral power of normal heartbeat intervals calculated in high frequency range Δf_{HF} (0.15 Hz-0.4 Hz) using the following formula:

$$HF = {\sum }_{j = {L}_{HFl}}^{{L}_{HFr}} {P}_{j} , ( {ms}^{2} )$$

where L_{HFl} and L_{HFr}—the frequency limits of the very high frequency range Δf_{HF} and Pⱼ—spectral values within this frequency range.

Once all required HRV parameters are calculated, the degree of tension (DT) of regulatory mechanisms and their functional reserve (FR) are calculated using the following canonical discriminant function equations:

DT=0.140×HR−0.165×SI−1.293×pNN50+0.623×HF%

FR=−0.112×HR−1.006×SI−0.047×pNN50−0.086×HF%

The obtained values of DT and FR are substituted to the respective discriminant function equations to identify four functional states (m1 to m4):

i) m1—pathological

ii) m2—premorbid

iii) m3—borderline

iv) m4—normal

The values of discriminant functions m1 to m4 are calculated using the following formulae:

m1=−26.195−4.9126×FR+0.0752×DT

m2=−4.1884−1.429×FR+0.718×DT

m3=−1.4238+0.0319×FR+0.7384×DT

m4=−1.7069+0.9762×FR−0.3592×DT

The obtained values of discriminant functions m1 to m4 may be used to calculate the values of posteriori probabilities pᵢ using the following formulae:

$${p}_{1} = \frac{{e}^{m 1}}{{e}^{m 1} + {e}^{m 2} + {e}^{m 3} + {e}^{m 4}}$$

$${p}_{2} = \frac{{e}^{m 2}}{{e}^{m 1} + {e}^{m 2} + {e}^{m 3} + {e}^{m 4}}$$

$${p}_{3} = \frac{{e}^{m 3}}{{e}^{m 1} + {e}^{m 2} + {e}^{m 3} + {e}^{m 4}}$$

$${p}_{4} = \frac{{e}^{m 4}}{{e}^{m 1} + {e}^{m 2} + {e}^{m 3} + {e}^{m 4}}$$

The calculated values of posteriori probabilities pᵢ will determine:
The risk of having a pathological state when p₁>p₂, p₃ and p₄;
The risk of having a premorbid state when p₂>p₁, p₃ and p₄;
The risk of having a borderline state when p₃>p₁, p₂ and p₄;
Normal state when p₄>p₁, p₂ and p₃.

This method of assessment of adaptation capabilities of the human organism and risks of disease development provides such assessment upon completion of recording of heartbeat intervals for a specific period of time.

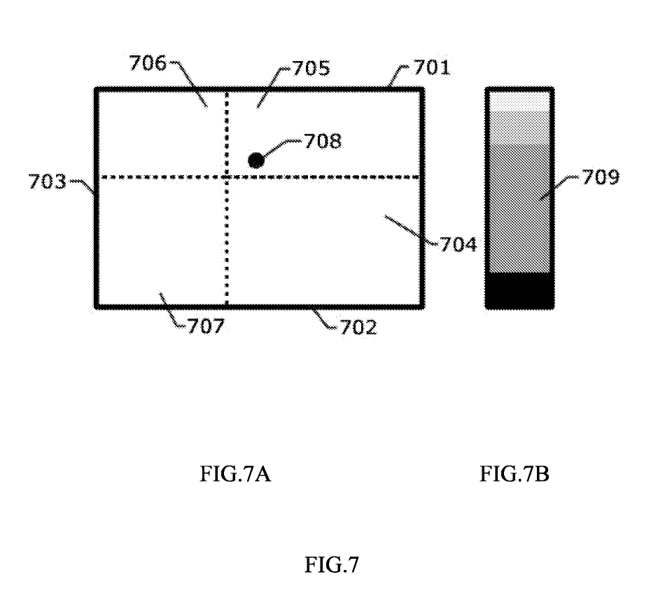

As best illustrated in FIG. 7, the results of this assessment should be visually presented in the form of a special diagram [702] with the orthogonal coordinates of Degree of Tension (DT) on the vertical scale [703] versus Functional Reserve (FR) on the horizontal scale [702]. In an exemplary embodiment of the present invention, the plot area of this diagram is divided by two orthogonal axes into four quadrants defining those four states: normal adaptation state [704], borderline state [705], premorbid state [706] and pathological state [707]. In another exemplary embodiment of the present invention, a resulting dot [708] is placed on the diagram in accordance with the respective values of DT and FR obtained from the measured data indicating the current condition of the mechanisms of physiological adaptation.

In yet another exemplary embodiment of the present invention, the obtained values of probabilities of the risk of being in either of those four states (p₁, p₂, p₃ and p₄) may be presented in the form of proportional bar diagram [709] since the sum of all probabilities is always equal to 100%. Each probability of respective state may be presented in specific color intuitively associated with the respective state. For example, normal state may be displayed in green while pathological state—in red.

When multiple testing with recording of heartbeat intervals for a specific period of time is performed in a regular manner over time (e.g. weekly), the evaluation of the dynamics of changes in the condition of the adaptation mechanisms and risks of having respective states may be done.

To perform the assessment of the dynamics of changes in adaptation mechanisms, it is very important to record heartbeat intervals data at the same time of the day (e.g. in the morning) to exclude the effect of circadian rhythms on body's regulatory mechanisms.

Such dynamics is evaluated using the standard methods of regression analysis, which can be implemented by a person having ordinary skill in the art. The results of this analysis (DT, FR, p₁, p₂, p₃, p₄) obtained in the course of repetitive use of this method of assessment are applied to specifically selected methods of linear or non-linear regression analysis to find respective linear or non-linear regression equations.

The obtained regression equations may allow for making conclusions about the dynamics of changes taking place—their degree and direction.

Depending on the number of assessments performed over time the level of statistical significance of such analysis can be estimated by means of calculation of confidence values for the obtained coefficients of regression equations. Such estimation can be implemented by a person having ordinary skill in the art.

Once the coefficients of linear or non-linear regression equations as well as their statistical significance values are obtained, mathematical time forecast (e.g. for one week ahead) of the estimated variables may be obtained using the methods of mathematical extrapolation of an appropriate order. Also the confidence levels values may be obtained for such forecast. This estimation can be implemented by a person having ordinary skill in the art.

This method of assessment of adaptation capabilities of the human organism and risks of disease development may be implemented in the form of various standalone smart devices or computer-based systems. Such devices and systems may be used by healthcare professionals and ordinary people. Healthcare professionals may use such systems in their clinics and offices or their mobile versions on the go visiting their clients at their workplace or testing people in public places during health fairs, etc. Ordinary people may use such devices and systems at their homes or as self-service kiosks located in public places. Moreover, such devices and systems may be used in special environments and situations like by military personnel at the battlefield, workers being in isolated and hard-to-reach locations, astronauts at orbital stations, etc.

A typical generic architecture of the computer-based system implementing the method of the present invention is best illustrated in FIG. 8, FIG. 10 AND FIG. 11. A measuring sensor, represented by a set of ECG lead wires with respective electrodes [801] provides input of an analog signal of heartbeat intervals as in step 101 of FIG. 10. The lead wires of electrodes [801] are connected to an electronic circuit [802]. The conditioning and analog filtering of the measured analog heart interval signals are done in electronic circuit [802] as in step 102 of FIG. 10. In another embodiment of the present invention, a measuring sensor may be represented by an optical PPG sensor [803] connected to an electronic circuit [804] providing input of an analog signal from PPG sensor [803], its conditioning and analog filtering. Further the analog signal, either ECG or PPG, is fed to an input of analog-to-digital converter [805] to convert it into digital format as in step 103 of FIG. 10. The digital heartbeat interval signal is then transmitted to a central processing unit (CPU) as in step 104 of FIG. 10. A central processing unit (CPU) [806] carries out overall control of the process of signal acquisition, its additional filtering by means of special digital filters, detects R-peaks in a digitized ECG signal, as in step 105, or specific fiduciary points on a pulse wave signal and calculates a series of heartbeat intervals between consecutive heartbeats as in step 106. The CPU [806] may place the original signal and obtained heartbeat intervals in the built-in random access memory (RAM) [807]. In another embodiment of the present invention, the system in the form of a standalone unit with built-in CPU chip providing capability to obtain heartbeat intervals without control from the external computer unit (desktop computer, portable computer, smartphone, tablet, etc.) this information may be stored in the built-in flash memory [808] for further transfer to an external computer unit by its request through a communication channel [809], which may be implemented in any of the following ways: wired interface such as USB, FireWire, Ethernet or wireless interface such as WiFi, Bluetooth, ZigBee, ANT and the like. In another embodiment of the present invention, utilizing an external computer unit, the obtained input signal and calculated heartbeat intervals may be transmitted directly to the external computer unit through the described communication interface [809] without storing this data in the flash memory [808]. The CPU [806] may receive requests and commands from the external computer unit via the communication interface [809] to control the process of data acquisition, processing and analysis as well as receive additional information needed to perform data analysis (e.g. height, weight, age). In an embodiment of the present invention, utilizing a standalone unit, without the necessity to be connected to an external computer unit, CPU [806] may perform data analysis and store the results in the flash memory [808]. In this embodiment, CPU [806] may receive the above mentioned information needed to perform this analysis as well as controlling commands from an input device [810] like functional keyboard or touch-screen monitor. In such embodiment the input signal, heartbeat intervals and the results of their analysis, calculated by the central processing unit [806], using the method of the present invention, as in steps 107 through 111 of FIGS. 10 and 11, may be shown on a built-in display [811] in symbolic or graphical format. The CPU [806] may also send this data to a printer [812] connected to the device or built in it.

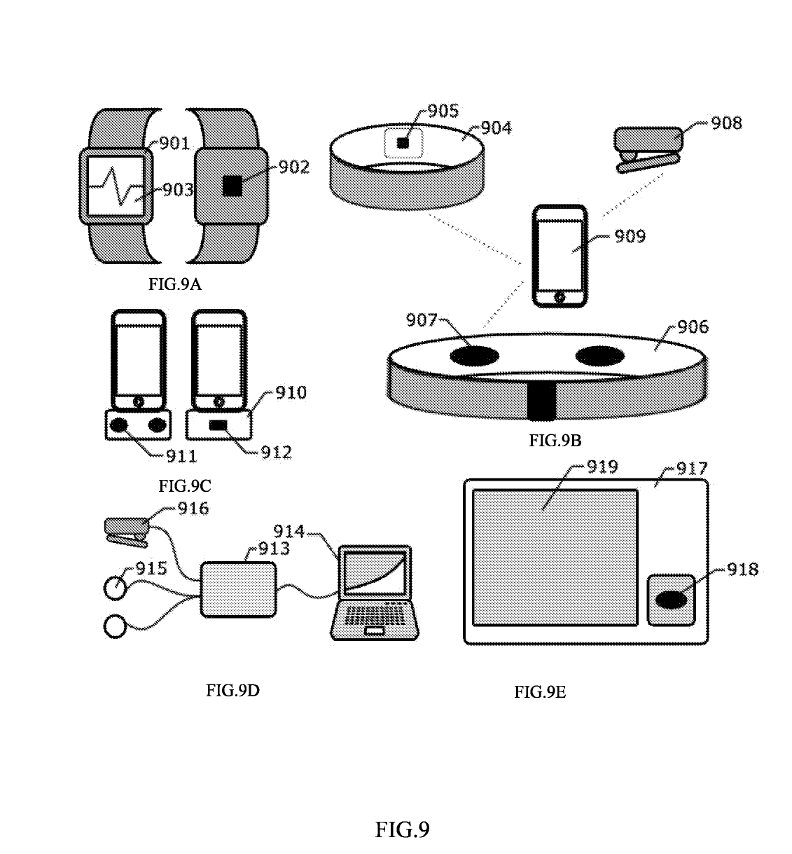

The present invention may have multiple implementations targeted to different ways of its use. Several possible options of such implementation are best illustrated in FIG. 9. One embodiment of the present invention may have a form factor of a wristwatch [901] wearable on wrists as best illustrated in FIG. 9A. It has a built-in optical PPG sensor [902] on the bottom side of the watch having direct contact with skin. The device may have a touch-screen display [903] to display data and provide input for necessary information and commands. Such device may be dedicated for personal use at home or on the go. The device may provide other handy functions like clock, calendar, alarm, media player, etc. With reference to FIG. 9B, another embodiment of the present invention may have form factors of (a) a bracelet [904] with a built-in PPG sensor [905] on its inner side wearable on wrists; (b) a chest strap [906] with built-in ECG electrodes [907] wearable on chest; (c) an elastic headband (not shown in figures) similar to a bracelet [904] wearable on a head where a PPG sensor is placed on a forehead; (d) a miniature ear-clip [908] wearable on earlobes. This device is only capable to acquire input signals, process them, store and transmit to an external computer unit [909] such as desktop computer, portable computer, smartphone, tablet by means of a wireless interface for further analysis, storage, display and interpretation. This device may be used by ordinary people at home. Another embodiment of the present invention may have a form factor of a device [910], as best illustrated in FIG. 9C, physically connected to a smartphone or a tablet forming one functional unit. This device may have built-in ECG electrodes [911] or a PPG sensor [912]. This device may be interfaced with a phone or tablet by means of physical connection to their input ports or wirelessly (e.g. via Bluetooth). This device may also be used by ordinary people at home. Another embodiment of this invention may have a form factor of a data acquisition device enclosed in a separate housing [913] and connected to an external computer [914] through a wired communication interface as best illustrated in FIG. 9D. The device may have external lead wires with ECG electrodes [915] or a PPG sensor in a finger-clip form factor [916]. The device is being controlled by a software program residing in the computer memory, which also carries out data collection, analysis, presentation and storage. This device may be used in professional settings like clinics, doctor's offices, laboratories, health check spot locations at work, etc. With reference to FIG. 9E, another embodiment of the present invention may have a form factor of a kiosk [917] having an embedded PPG sensor [918] and touch-screen display [919]. Inside the kiosk there is a computer module with a hard-wired connection to a PPG sensor. This device may be used for the purpose of self-assessment by ordinary people at various locations such as drugstores, general stores, waiting rooms, etc.

Additionally, other variations are within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of health risk assessment by means of measuring and evaluating the functional state of the mechanisms of physiological adaptation of human organism, the method comprising:
recording analog signals of heartbeat intervals with positioning of at least one heartbeat recording sensor at specific location;
filtering said analog signals of heartbeat intervals by at least one filtering circuit;
converting said analog signals to digital signals of heartbeat intervals by said analog to digital signal converter;
transmitting said digital signals of heartbeat intervals by said analog to digital signal converter to at least one central processing unit;
detecting R-peaks from said digital signals of heartbeat intervals by said at least one central processing unit;
measuring time intervals between two consecutive said R-peaks by said central processing unit;
calculating heartbeat variability parameters from said heartbeat intervals by said central processing unit;
calculating degree of tension (DT) of regulatory mechanisms and their functional reserve (FR) by said central processing unit based on said heartbeat variability parameters;
calculating plurality of functional states based on said DT and FR by said processor; and
calculating posteriori probabilities based on plurality of functional states by said central processing unit;
wherein probabilities of health risk is determined based on values of said degree of tension (DT) of regulatory mechanisms, said functional reserve (FR), said at least one functional state and said posteriori probabilities and said health risk is displayed through at least one diagram on a display device.

2. The method of health risk assessment by means of measuring and evaluating the functional state of the mechanisms of physiological adaptation of human organism as in claim 1, wherein said at least one heartbeat recording sensor is an electrocardiogram (ECG) sensor.

3. The method of health risk assessment by means of measuring and evaluating the functional state of the mechanisms of physiological adaptation of human organism as in claim 1, wherein said at least one heartbeat recording sensor is a photoplethysmograph (PPG) sensor.

4. The method of health risk assessment by means of measuring and evaluating the functional state of the mechanisms of physiological adaptation of human organism as in claim 1, wherein said recording of analog signals of heartbeat intervals with said heartbeat recording device is done for a period of 5 minutes.

5. The method of health risk assessment by means of measuring and evaluating the functional state of the mechanisms of physiological adaptation of human organism as in claim 1, wherein said recording of analog signals of heartbeat intervals with said heartbeat recording device is done for a period of less than 5 minutes.

6. The method of health risk assessment by means of measuring and evaluating the functional state of the mechanisms of physiological adaptation of human organism as in claim 1, wherein said recording of analog signals of heartbeat intervals with said heartbeat recording device is done for a period of more than 5 minutes.

7. The method of health risk assessment by means of measuring and evaluating the functional state of the mechanisms of physiological adaptation of human organism as in claim 1, wherein said transmission of digital signals of heartbeat intervals by said analog to digital signal converter to at least one central processing unit is done through wireless system.

8. The method of health risk assessment by means of measuring and evaluating the functional state of the mechanisms of physiological adaptation of human organism as in claim 1, wherein said transmission of digital signals of heartbeat intervals by said analog to digital signal converter to at least one central processing unit is done through wires.

9. The method of health risk assessment by means of measuring and evaluating the functional state of the mechanisms of physiological adaptation of human organism as in claim 1, wherein said heartbeat variability parameters comprise Heartbeat (HR), Stress Index (SI), pNN50 and HF %.

10. The method of health risk assessment by means of measuring and evaluating the functional state of the mechanisms of physiological adaptation of human organism as in claim 9, wherein said heartbeat variability parameter Heartbeat (HR) is mean value of all time said heartbeat intervals measured between two said consecutive R-peaks.

11. The method of health risk assessment by means of measuring and evaluating the functional state of the mechanisms of physiological adaptation of human organism as in claim 9, wherein said heartbeat variability parameter Stress Index (SI) is an index of a stress level and is calculated in accordance with the following equations:
$$SI = \frac{AMo}{2 Mo Δ X}$$
wherein Mo is mode which is the most frequently observed said heartbeat interval value in a series of said recorded heartbeat intervals in seconds, AMo is an amplitude of said mode Mo and is the total number of said heartbeat intervals of said recorded heartbeats corresponding to the said mode value calculated as percentage of the total number of said heartbeat intervals of said recorded heartbeats and ΔX is the difference between maximal and minimal values of a series of said heartbeat intervals of said recorded heartbeats measured in seconds.

12. The method of health risk assessment by means of measuring and evaluating the functional state of the mechanisms of physiological adaptation of human organism as in claim 9, wherein said heartbeat variability parameter pNN50 is percentage of the number of consecutive pairs of normal heartbeat intervals having difference of 50 or more milliseconds to the total number of consecutive pairs of normal heartbeat intervals of the recorded.

13. The method of health risk assessment by means of measuring and evaluating the functional state of the mechanisms of physiological adaptation of human organism as in claim 9, wherein said heartbeat variability parameter HF % is percentage ratio of the spectral power of normal heartbeat intervals in the high frequency range calculated using the following equation:
$$HF \% = 100 \times \frac{HF}{TP}$$
wherein,
TP is the sum of spectral powers of normal heartbeat intervals calculated in all frequency ranges using the following formula:
TP=VLF+LF+HF
wherein,
VLF is spectral power of normal heartbeat intervals calculated in very low frequency range Δf_{VLF} (0.0033 Hz-0.04 Hz) using the following formula:
$$VLF = {\sum }_{j = {L}_{VLFl}}^{{L}_{VLFr}} {P}_{j} , ( {ms}^{2} )$$
wherein, L_{VLFl} and L_{VLFr} are the frequency limits of the very low frequency range Δf_{VLF} and Pⱼ spectral values within this frequency range,
wherein, LF is spectral power of normal heartbeat intervals calculated in low frequency range Δf_{LF} (0.04 Hz-0.15 Hz) using the following formula:
LF=Σ_{j=L}_{_{FLl}}^{L}^{_{LFr}}Pⱼ,(ms²)
wherein L_{LFl} and L_{LFr} are the frequency limits of the low frequency range Δf_{LF} and Pⱼ is the spectral values within this frequency range,
wherein HF is the spectral power of normal heartbeat intervals calculated in high frequency range Δf_{HF} (0.15 Hz-0.4 Hz) using the following formula:
HF=Σ_{j=L}_{_{HLl}}^{L}^{_{HFr}}Pⱼ,(ms²)
wherein L_{HFl} and L_{HFr} are the frequency limits of the very high frequency range Δf_{HF} and Pⱼ is spectral values within this frequency range.

14. The method of health risk assessment by means of measuring and evaluating the functional state of the mechanisms of physiological adaptation of human organism as in claim 9, wherein said degree of tension (DT) of regulatory mechanisms and said functional reserve (FR) are calculated using the following equations:
DT=0.140×HR−0.165×SI−1.293×pNN50+0.623×HF%
FR=−0.112×HR−1.006×SI−0.047×pNN50−0.086×HF%

15. The method of health risk assessment by means of measuring and evaluating the functional state of the mechanisms of physiological adaptation of human organism as in claim 9, wherein said functional states include pathological (m1), premorbid (m2), borderline (m3) and normal (m4) states.

16. The method of health risk assessment by means of measuring and evaluating the functional state of the mechanisms of physiological adaptation of human organism as in claim 15, wherein said functional states pathological (m1), premorbid (m2), borderline (m3) and normal (m4) states are calculated using the following equations:
m1=−26.195−4.9126×FR+0.0752×DT
m2=−4.1884−1.429×FR+0.718×DT
m3=−1.4238+0.0319×FR+0.7384×DT
m4=−1.7069+0.9762×FR−0.3592×DT

17. The method of health risk assessment by means of measuring and evaluating the functional state of the mechanisms of physiological adaptation of human organism as in claim 15, wherein said plurality of posteriori probabilities are calculated using the following formulae:
$${p}_{1} = \frac{{e}^{m 1}}{{e}^{m 1} + {e}^{m 2} + {e}^{m 3} + {e}^{m 4}}$$
$${p}_{2} = \frac{{e}^{m 2}}{{e}^{m 1} + {e}^{m 2} + {e}^{m 3} + {e}^{m 4}}$$
$${p}_{3} = \frac{{e}^{m 3}}{{e}^{m 1} + {e}^{m 2} + {e}^{m 3} + {e}^{m 4}}$$
$${p}_{4} = \frac{{e}^{m 4}}{{e}^{m 1} + {e}^{m 2} + {e}^{m 3} + {e}^{m 4}}$$

18. The method of health risk assessment by means of measuring and evaluating the functional state of the mechanisms of physiological adaptation of human organism as in claim 17, wherein said calculated values of posteriori probabilities determine:
the risk of having a pathological state when p₁>p₂, p₃ and p₄;
the risk of having a premorbid state when p₂>p₁, p₃ and p₄;
the risk of having a borderline state when p₃>p₁, p₂ and p₄; and
normal state when p₄>p₁, p₂ and p₃.

19. The method of health risk assessment by means of measuring and evaluating the functional state of the mechanisms of physiological adaptation of human organism as in claim 1, wherein said at least one diagram displayed on a said display device is a diagram with the orthogonal coordinates of said Degree of Tension (DT) on a vertical scale versus said Functional Reserve (FR) on a horizontal scale with plot of said diagram divided by two orthogonal axes into four quadrants defining said plurality functional states and a resulting dot is placed on said diagram in accordance with respective values of said DT and said FR indicating current condition of said mechanisms of physiological adaptation.

20. The method of health risk assessment by means of measuring and evaluating the functional state of the mechanisms of physiological adaptation of human organism as in claim 1, wherein said at least one diagram is presented in the form of a proportional bar diagram with the sum of all said plurality of posteriori probabilities equal to 100%.

21. The method of health risk assessment by means of measuring and evaluating the functional state of the mechanisms of physiological adaptation of human organism as in claim 20, wherein each said probabilities is presented in specific color in at least said one diagram presented in the form of said proportional bar diagram with the sum of all said plurality of posteriori probabilities equal to 100%.

22. The method of health risk assessment by means of measuring and evaluating the functional state of the mechanisms of physiological adaptation of human organism as in claim 1, wherein said specific location for positioning of said at least one heartbeat recording sensor is wrist, chest, earlobe, toe, arms and forehead.

23. The method of health risk assessment by means of measuring and evaluating the functional state of the mechanisms of physiological adaptation of human organism as in claim 1, wherein measuring of the heartbeat intervals are performed periodically to carry out an assessment of the dynamics of adaptation capabilities of the human organism and risks of development of said plurality of functional states and results of said dynamics is evaluated using standard methods of regression analysis.

24. The method of health risk assessment by means of measuring and evaluating the functional state of the mechanisms of physiological adaptation of human organism as in claim 23, wherein said results of analysis obtained in the course of repetitive use of said method of assessment are applied to specifically selected methods of linear and non-linear regression analysis to find respective linear and non-linear regression equations and said obtained regression equations allow for making conclusions about the dynamics of changes taking place and their degree and direction and depending on the number of assessments performed over time the level of statistical significance of such said analysis can be estimated by means of calculation of confidence values for said obtained coefficients of regression equations.

25. An apparatus for health risk assessment by means of measuring and evaluating the functional state of the mechanisms of physiological adaptation of human organism, the apparatus comprising:
at least one sensor for recording analog signals of heartbeat intervals positioned at specific location;
at least one filtering circuit for filtering said analog signals of heartbeat intervals;
at least one analog to digital signal converter for converting said analog signals to digital signals of heartbeat intervals;
at least one communication means for transmitting said digital signals of heartbeat intervals;
at least one central processing unit for receiving said digital signals of heartbeat intervals, detecting R-peaks from said digital signals of heartbeat intervals, measuring time intervals between two consecutive said R-peaks, calculating heartbeat variability parameters from said time intervals of heartbeat intervals, calculating degree of tension (DT) of regulatory mechanisms and their functional reserve (FR), calculating plurality of functional states based on said DT and FR by said processor and calculating posteriori probabilities based on plurality of functional states by said central processing unit; and
at least one display device for displaying said plurality of functional states and probabilities.
